(19) United States
(12) Patent Application Publication
(10) Pub. No.: US 2005/0191245 A1
(43) Pub. Date: Sep. 1, 2005

(54) Nasal administration of calcium channel, blockers for treatment of hypertension and other cardiovascular disorders

(76) Inventors: Christopher P. Adams, Somerville, MA (US); Timothy Maher, Milton, MA (US); Karl P. Weinrich, Bridgewater, MA (US)

Correspondence Address: REED INTELLECTUAL PROPERTY LAW GROUP, 1400 PAGE MILL ROAD, PALO ALTO, CA, 94304-1124, US

(21) Appl. No.: 10/789,965

(22) Filed: Feb. 27, 2004

(51) Int. Cl. A61L 9/04; A61K 9/14

(52) U.S. Cl. 424/45; 424/46

(57) ABSTRACT

Pharmaceutical compositions useful for the treatment of cardiovascular disorders such as hypertension are described. These compositions are formulated for nasal administration and comprise a therapeutically effective amount of a calcium channel blocker, optionally a pharmaceutically acceptable carrier that is suitable for nasal administration, and one or more optional members selected from excipients and additional pharmaceutically active agents.

### TECHNICAL FIELD

This invention relates to methods of using nasal pharmaceutical compositions for treating cardiovascular disorders such as hypertension. More particularly, the invention relates to methods of using nasal compositions comprising a calcium channel blocker. In addition, the invention relates to pharmaceutical compositions, and more particularly relates to pharmaceutical compositions comprising a therapeutically effective amount of a calcium channel blocker, optionally a pharmaceutically acceptable carrier that is suitable for nasal administration, and one or more optional members selected from excipients and additional pharmaceutically active agents.

### BACKGROUND

Blood pressure is measured as the pressure of blood in the arteries when the heart beats (systolic) over the pressure between heartbeats (diastolic). High blood pressure, or hypertension, is generally considered to be a pressure greater than or equal to 140 mm Hg systolic and/or greater than or equal to 90 mm Hg diastolic. Hypertension, a risk factor for heart disease and stroke, is serious, but treatable.

Typically, hypertension occurs when the smaller blood vessels in the body narrow or become stiff and less compliant, which causes the blood to exert excessive pressure against the vessel walls. The heart must therefore work harder to maintain adequate blood flow, but at a higher pressure. Although the body can tolerate increased blood pressure for a period of time, eventually the heart can become enlarged and the heart along with other organs such as the kidneys, brain and eyes can be damaged. Hypertension is dangerous because it often does not present symptoms.

The most common type of hypertension is referred to as essential, or primary, hypertension, which is when a physician is unable to identify a specific cause. Genetic factors appear to play a major role in essential hypertension. Another type of hypertension, secondary hypertension, however, has recognizable causes, which are usually treatable. Causes of secondary hypertension include certain medical conditions, medications, excessive use of alcohol or caffeine, use of cocaine, long-term consumption of large amounts of licorice, stress and smoking. Some of the medical conditions that contribute to secondary hypertension are renal parenchymal disease, primary aldosteronism, hyperthyroidism, myxedema, coarctation of the aorta, renovascular disease, pregnancy, cirrhosis, kidney disease, and Cushing's disease. Certain drugs such as oral contraceptives, sympathomimetics (e.g., pseudoephedrine), corticosteroids, and indomethacin can cause secondary hypertension.

It is estimated that only a small number of hypertensive patients have their blood pressure under control or are even aware that they have hypertension. While life style changes such as extra rest, stress reduction, exercise, moderate weight reduction, and dietary sodium restriction, can be used to treat patients with mild hypertension and no heart disease, antihypertensive drug therapy is often a more effective treatment. Aggressive drug treatment of long-term hypertension can significantly reduce mortality and morbidity from heart disease and other causes in both men and women. For more severe hypertension or for mild cases that do not respond to changes in diet and lifestyle, drug treatment is usually necessary. A single-drug regimen can often control mild to moderate hypertension. More severe hypertension, however, often requires a combination of two or more drugs.

There are a number of antihypertensive drugs on the market. These drugs usually fall into the following categories: (1) diuretics (e.g., chlorothiazide, spironolactone), which cause the body to excrete water and salt; (2) β-blockers (e.g., acebutolol, atenolol, betaxolol, bisprolol, metoprolol), which block the effects of epinephrine and norepinephrine, thus easing the heart's pumping action and via indirect mechanisms relaxing blood vessels; (3) calcium channel blockers (e.g., verapamil, diltiazem, nifedipine, amlodipine, felodipine, isradipine, nicardipine, nisoldipine), which help decrease the contractions of the heart and widen blood vessels (4) ACE inhibitors (e.g., benazepril, captopril, enalapril, lisinopril, moexipril, quinapril, ramipril, trandolapril), which reduce the production of angiotensin II, a chemical that causes arteries to constrict; (5) angiotensin II receptor blockers (e.g., irbesartan, losartan, valsartan), which interfere with the renin-angiotensin system; (6) sympatholytics including centrally acting a agonists (e.g., clonidine, guanabenz, guanfacine, methyldopa), α₁-adrenergic blockers (e.g., doxazosin, prazosin, terazosin,) and peripheral-acting adrenergic ganglionic blockers (e.g., guanadrel sulfate, guanethidine, reserpine); and (7) vasodilators (e.g., minoxidil, hydralazine), which dilate or relax blood vessels.

Research has indicated that diuretics, ACE inhibitors, and beta-blockers all reduce the risk for fatal and nonfatal cardiovascular events. As first-line treatment for most patients with hypertension who have no complicating factors, physicians generally recommend that they use blockers or diuretics. Currently, many known drugs used to treat hypertension have adverse side effects (e.g., diuretics can cause sexual dysfunction, adverse metabolic effects, and/or breast tenderness; β-blockers can cause sexual dysfunction and/or adverse central nervous system effects; ACE inhibitors can cause a dry irritating cough) or are contraindicated for particular patients (e.g. β-blockers are contraindicated for patients with severe asthma; calcium channel blockers are contraindicated for patients with left ventricular dysfunction), which can make on-going treatment and compliance difficult for some patients.

In view of the foregoing, it will be appreciated that providing improved antihypertensive drug formulations and methods of their use would provide a significant advancement in the art. In the present invention, calcium channel blockers are nasally administered so as to increase the amount of drug delivered to the brain of a patient, decrease the peripheral exposure to the drug, and to decrease the time required for the drug to reach the brain.

### SUMMARY OF THE INVENTION

One aspect of the invention relates to a method of increasing the amount of a calcium channel blocker delivered to the brain of a patient suffering from hypertension, comprising nasally administering a pharmaceutical composition comprising a therapeutically effective amount of the calcium channel blocker.

Another aspect of the invention relates to a method of treating a patient suffering from hypertension comprising targeting delivery of a calcium channel blocker to the brain of the patient by administering the calcium channel blocker intranasally.

Yet another aspect of the invention relates to an improved method of treating a cardiovascular disorder, such as hypertension, with a calcium channel blocker; the improvement comprising administering the calcium channel blocker nasally to maximize the amount of calcium channel blocker reaching the brain and to minimize peripherally mediated adverse events.

Still another aspect of the invention relates to a pharmaceutical composition formulated for nasal drug administration, comprising a therapeutically effective amount of a calcium channel blocker selected from cinnarizine, and nifedipine.

Additional aspects, advantages and features of the invention will be set forth, in part, in the description that follows, and, in part, will become apparent to those skilled in the art upon examination of the following, or may be learned by practice of the invention.

### BRIEF DESCRIPTION OF THE DRAWINGS

FIG. 1 shows the effect of intranasal administration of diltiazem on mean arterial blood pressure.

FIG. 2 shows the comparative effects of intranasal administration of different calcium blockers (i.e., cinnarizine and nifedipine) on mean arterial blood pressure.

FIG. 3 shows HPLC detection of diltiazem in striatal microdialysates before and after intranasal administration.

FIG. 4 shows the effect of intranasal administration of diltiazem on the total distance traveled by rats in an open field locomotion test.

FIG. 5 shows the comparative effects of intranasal administration of different calcium channel blockers (i.e., verapamil and diltiazem) on the total distance traveled by rats in an open field locomotion test.

FIG. 6 shows the comparative effects of intranasal administration of different calcium channel blockers (i.e., cinnarizine and nifedipine) on the total distance traveled by rats in an open field locomotion test.

### DETAILED DESCRIPTION OF THE INVENTION

### I. DEFINITIONS

Before describing the present invention in detail, it is to be understood that this invention is not limited to particular drug delivery systems, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Thus, for example, reference to “a calcium channel blocker” includes a combination of two or more calcium channel blockers, reference to “a pharmaceutically acceptable carrier” includes combinations of two or more pharmaceutically acceptable carriers, reference to “an excipient” includes combinations of two or more excipients, and the like.

In describing and claiming the present invention, the following terminology will be used in accordance with the definitions set forth below.

The terms “active agent,” “drug” and “pharmacologically active agent” are used interchangeably herein to refer to a chemical material or compound which, when administered to an organism (human or animal) induces a desired pharmacologic effect. Included are derivatives that include pharmacologically acceptable and pharmacologically active salts, esters and amides, as well as prodrugs, conjugates and active metabolites. Analogs of those compounds or classes of compounds specifically mentioned that also induce the desired pharmacologic effect, are also included.

By “pharmaceutically acceptable carrier” is meant a material or materials that are suitable for intranasal drug administration and are not biologically or otherwise undesirable, i.e., that may be administered to a patient along with an active agent without causing any undesirable biological effects or interacting in a deleterious manner with any of the other components of the pharmaceutical formulation in which it is contained. Typically, the material (e.g., carrier or excipient) has met the required standards of toxicological and manufacturing testing or it is included on the Inactive Ingredient Guide prepared by the U.S. Food and Drug administration.

Similarly, a “pharmacologically acceptable” salt, ester, amide or other derivative of an active agent as provided herein is a salt, ester, amide or other derivative that is not biologically or otherwise undesirable.

By the terms “effective amount” or “therapeutically effective amount” of an active agent as provided herein are meant a nontoxic, but sufficient amount of the agent to provide the desired therapeutic effect. The exact amount required will vary from subject to subject, depending on the age, weight, and general condition of the subject, the severity of the condition being treated, the judgment of the clinician, and the like. Thus, it is not always possible to specify an exact “effective amount.” However, an appropriate “effective” amount in any individual case may be determined by one of ordinary skill in the art using routine experimentation.

The term “dosage form” denotes any form of a pharmaceutical composition that contains an amount of active agent sufficient to achieve a therapeutic effect with a single administration. The frequency of administration that will provide the most effective results in an efficient manner without overdosing will vary with the characteristics of the particular active agent, including both its pharmacological characteristics and its physical characteristics, such as hydrophilicity.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, the present method of “treating” hypertension, as the term “treating” is used herein, encompasses both prevention of hypertension in a predisposed patient and treatment of hypertension in a clinically symptomatic patient.

The terms “condition,” “disease” and “disorder” are used interchangeably herein as referring to a physiological or pathophysiological state that can be prevented or treated by administration of a pharmaceutical formulation as described herein.

The term “patient” as in treatment of “a patient” refers to a mammalian individual afflicted with or prone to a condition, disease or disorder as specified herein, and includes both humans and animals.

The terms “nasal drug administration” and “intranasal drug administration” are used interchangeably herein as referring to an active agent is applied to, or introduced into, the nostrils of a patient such that the drug contacts tissues in the nasal passages, such as the nasal mucosa or sinuses, from which the drug is absorbed into the body. The resulting therapeutic effect is primarily a result of topical absorption or infusion into tissues to which the drug is administered. Thus, the phrase “nasal drug administration” includes administering the formulations described herein to any part, tissue or organ within a patient that is directly or indirectly involved with either the external and internal nasal passages, or involved with the nasal sinuses.

“Optional” or “optionally” means that the subsequently described event or circumstance may or may not occur, and that the description includes instances where said event or circumstance occurs and instances where it does not. For example, reference to an “optional member” in a formulation indicates that such a member may or may not be present, and the description includes formulations wherein a member is present and formulations wherein a member is not present.

### II. PHARMACEUTICAL COMPOSITIONS

As noted above, in one embodiment, the invention is a pharmaceutical composition formulated for nasal drug administration, comprising a therapeutically effective amount of a calcium channel blocker. In another embodiment, the invention is a pharmaceutical composition formulated for nasal drug administration, comprising a therapeutically effective amount of a calcium channel blocker and a pharmaceutically acceptable carrier that is suitable for nasal drug administration. The pharmaceutical compositions of the present invention may take any form suitable for delivering the active agent to the nasal passages of a patient. For example, the composition may be formulated in an aerosol, as a liquid, as a dry powder form, and so forth. A therapeutically effective amount of one or more additional pharmaceutically active agents may also be included.

The compositions of the present invention may also contain various excipients, provided such excipients do not have a deleterious effect on the intended patient or have a deleterious chemical or physical effect on any component in the composition. Thus, for example, excipients such as permeation enhancers, bioadhesive materials, preservatives, moisturizing agents, surface active agents, buffering agents, suspending agents, and the like can be combined with the composition. The type and amount of any excipient will depend on the type of formulation and the device used for administration, as will be appreciated by one of ordinary skill in the art. Specific examples of each of these excipients are well known by those skilled in the art of pharmaceutical formulation.

A. Active Agents

Any of the active agents in the formulation may be administered in the form of a pharmacologically acceptable salt, ester, amide, prodrug or derivative or as a combination thereof. Salts, esters and derivatives of the active agents may be prepared using standard procedures known to those skilled in the art of synthetic organic chemistry and described, for example, by J. March, “Advanced Organic Chemistry: Reactions, Mechanisms and Structure,” 4th Ed. (New York: Wiley-Interscience, 1992). For example, acid addition salts are prepared from the free base (e.g., compounds having a neutral —NH₂ or cyclic amine group) using conventional means, involving reaction with a suitable acid. Typically, the base form of an active agent is dissolved in a polar organic solvent such as methanol or ethanol and the acid is added at a temperature of about 0 to 100° C., preferably at ambient temperature. The resulting salt either precipitates or may be brought out of solution by addition of a less polar solvent. Suitable acids for preparing the acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like as well as inorganic acids, e.g., hydrochloric acid, hydrobromic acid, sulfuric acid, nitric acid, phosphoric acid, and the like. An acid addition salt may be reconverted into the free base by treatment with a suitable base. Basic addition salts of an active agent having an acid moiety (e.g., carboxylic acid group or hydroxyl group) are prepared in a similar manner using a pharmaceutically acceptable base. Suitable bases include both inorganic bases, e.g., sodium hydroxide, potassium hydroxide, ammonium hydroxide, calcium hydroxide, magnesium hydroxide, and the like, as well as organic bases such as trimethylamine, and the like. Preparation of esters involves functionalization of hydroxyl and/or carboxyl groups that may be present within the molecular structure of the drug. The esters are typically acyl-substituted derivatives of free alcohol groups, i.e., moieties which are derived from carboxylic acids of the formula RCOOH where R is alkyl, and preferably is lower, i.e., C₁₋₆ alkyl. Esters can be reconverted to the free acids, if desired, by using conventional hydrogenolysis or hydrolysis procedures. Preparation of amides and prodrugs can be carried out in an analogous manner. Other derivatives of the active agents may be prepared using standard techniques known to those skilled in the art of synthetic organic chemistry, or may be deduced by reference to the pertinent literature and texts.

Stereoisomers of the active agents are also included as part of the formulations described herein. A stereoisomer is a compound having the same molecular weight, chemical composition, and constitution as another, but with certain atoms arranged differently. That is, certain identical chemical moieties are at different orientations in space. This difference usually has the consequence of rotating the plane of polarized light in a differential manner. A pair of stereoisomers that are mirror images of each other are defined as enantiomers. Individual stereoisomers or enantiomers may have unique or beneficial properties that make that individual isomer particularly well suited for the present invention. Consequently, individual stereoisomers or enantiomers and mixtures thereof of the active agents are included as part of the invention. Thus, each active agent may be present in the formulation as a racemate, i.e., equal amounts of each enantiomer, an enantiomerically pure form, or a mixture of nonequal amounts of each enantiomer.

The various hydrates of the active agents are also included in the formulations of the invention. As is known, one or more water molecules may associate with a particular compound based on, for example, the availability of hydrogen bonding. Methods of producing hydrated species are known and include, for example, placing the active agent in a humid environment. In addition, methods of removing one or more water molecules are known and include, by way of example, exposing the active agent to dry heat.

1. Calcium Channel Blockers

The compositions of this invention are not limited to one calcium channel blocker as combinations of calcium channel blockers may also be present. Calcium channel blockers for use in the methods of the invention include by way of illustration, and not limitation, verapamil, diltiazem, nifedipine, amlodipine, felodipine, isradipine, nicardipine, cinnarizine, and nisoldipine. In a specific embodiment, the calcium channel blocker useful in the composition of the invention is selected from cinnarizine and nifedipine. The calcium channel blocker may be present in the composition as a salt, ester, amide, prodrug, or other derivative, or may be functionalized in various ways as will be appreciated by those skilled in the art.

The weight percentage of calcium channel blocker in the compositions is typically from 0.2% to about 30% by weight, more preferably from about 2% to about 20% by weight of the composition. Typically, compositions according to the present invention will contain a unit dose of from about 0.2 to about 20 mg of a calcium channel blocker per dose.

2. Additional Active Agents

The compositions may further comprise one or more additional pharmaceutically active agents applied in the treatment of hypertension besides the well-known auxiliary agents. Such other active agents can be, for instance diuretics such as metolazone, indapamide, benzothiadiazines and thiazide type diuretics (e.g. hydroflumethiazide and chlorthalidone) and potassium-sparing diuretics (e.g. triamterene); P-blockers such as acebutolol, alprenolol, atenolol, betaxolol, bevantol, bisprolol, bupranolol, bunitrolol, exaprolol, indenolol, indanolol, labetalol, metoprolol, moprolol, nifenalol, nitrolol, oxprenolol, pamatolol, penbutolol, pronethalol, propranolol, pargolol, procinolol, practolol, sotalol, triprenolol, tolamolol, toliprolol, and timolol; ACE inhibitors such as benazepril, captopril, enalapril, lisinopril, moexipril, quinapril, ramipril, trandolapril; angiotensin II receptor blockers such as irbesartan, losartan, valsartan; adrenergic inhibitors such as centrally acting a agonists (e.g., clonidine, guanabenz, guanfacine, methyldopa), a₁-adrenergic blockers (e.g., doxazosin, prazosin, terazosin,) and peripheral-acting adrenergic blockers (e.g., guanadrel sulfate, guanethidine, reserpine); and vasodilators such as minoxidil, hydralazine.

B. Pharmaceutically Acceptable Carrier

1. Aerosol Formulations

The formulations of the present invention may take the form of an aerosol composition for inhalation. Aerosol formulations are known to those skilled in the art and are described in Remington: The Science and Practice of Pharmacy, supra. Briefly, the aerosol formulation of the invention is either a solution aerosol in which the active agent is soluble in the carrier (e.g., propellant) and optional solvent, or is a dispersion aerosol in which the active agent is suspended or dispersed throughout the carrier and optional solvent.

The carrier in the aerosol formulations of the invention is generally a propellant, usually a compressed gas, e.g., air, nitrogen, nitrous oxide, and CO₂, a mixture of compressed gases, a liquefied gas or a mixture of liquefied gases. A mixture of propellants, when present in the formulations, may be comprised of two, three, or four propellants. Preferred mixtures of propellants, however, comprise only two propellants. Any propellant used in the art of preparing aerosol formulations may be used herein.

Typically, the propellant is a chlorofluorocarbon, a hydrochlorofluorocarbon, a hydrogen-containing fluorocarbon, a perfluorocarbon, a hydrocarbon or a mixture thereof. Preferably, the propellant is a hydrochlorofluorocarbon, a hydrogen-containing fluorocarbon, a perfluorocarbon or a mixture thereof.

Preferred chlorofluorocarbons include dichlorotetrafluoroethanes (e.g., CClF₂CClF₂, CCl₂FCF₃), trichloromonofluoromethane, dichlorodifluoromethane, chloropentafluoroethane, and mixtures thereof. Preferred hydrochlorofluorocarbons include monochlorodifluoromethane, monochlorodifluoroethane (e.g., 1-chloro-1,1-difluoroethane), and mixtures thereof. Preferred hydrogen-containing fluorocarbons include C₁₋₄ hydrogen-containing fluorocarbons such as CHF₂CHF₂, 1,1,1,2-tetrafluoroethane (HFA-134a), difluoroethane (e.g., 1,1-difluoroethane), 1,1,1,2,3,3,3-heptafluoropropane (HFA-227), and mixtures thereof. Preferred perfluorocarbons include CF₃CF₃, CF₃CF₂CF₃, octafluorocyclobutane, and mixtures thereof. Preferred hydrocarbons include propane, isobutane, n-butane, dimethyl ether, and mixtures thereof Most preferably, the propellant is selected from the group consisting of difluoroethane, CHF₂CHF₂, 1,1,1,2-tetrafluoroethane, 1,1,1,2,3,3,3-heptafluoropropane, CF₃CF₃, CF₃CF₂CF₃, octafluorocyclobutane, and mixtures of any of the foregoing.

As will be appreciated by one skilled in the art, the aerosol formulations of the invention may include one or more excipient. For example, the aerosol formulations may contain: a solvent (e.g., water, ethanol and mixtures thereof) for increasing the solubility of the active agent; an antioxidant (e.g., ascorbic acid) for inhibiting oxidative degradation of the active agents; a dispersing agent (e.g., sorbitan trioleate, oleyl alcohol, oleic acid, lecithin, e.g., soya lecithin, corn oil, or combinations thereof) for preventing agglomeration of particles; and/or a lubricant (e.g., isopropyl myristate) for providing slippage between particles and lubricating the components (e.g., the valve and spring) of the inhaler.

As described below with respect to dry powder formulations, the particle size released from aerosol formulations must be appropriate for nasal administration. Solution aerosols inherently produce small particles upon actuation of the inhaler given that the active agent is expelled along with the carrier, i.e., propellant, solution as it evaporates. Consequently, solution aerosols produce sufficiently small particles, e.g., within a range of about 0.1 to about 65μm, of active agents upon administration. In contrast, dispersion aerosols contain undissolved active agents in which particle size remains constant, i.e., the size of the particles in the dispersion aerosol remains unchanged as the active agent is delivered to the patient. Thus, the active agents should have an appropriate particle size before being formulated into a dispersion aerosol. Consequently, methods of reducing the particle size of the active agents for the dry powder formulations described below are equally applicable for preparing active agents with an appropriate particle size in a dispersion aerosol. Furthermore, the same ranges of particle sizes preferred for the dry powder formulations are equally applicable for dispersion aerosols.

The aerosol formulation may be prepared by employing a cold filling process. Initially, the components of the aerosol formulation and an aerosol container are cooled, e.g., to about −40° C., such that the carrier, i.e., propellant, is a liquid. All components except for the carrier are placed into the aerosol container. Thereafter, the carrier is added, the components are mixed, and a valve assembly is inserted into place. The valve assembly is then crimped such that the container is airtight. Thereafter, the container and formulation contained therein are allowed to return to ambient temperature.

As an alternative to the cold filling process, the aerosol formulations may be prepared by transferring a carrier from a bulk container. In such a process, the components, except for the carrier, are initially placed into an empty aerosol container. A valve assembly is then inserted and crimped into place. The carrier, under pressure and in liquid form, is metered through the valve assembly from a bulk container or tank of carrier. The container housing the formulation is checked to ensure that the pressurized contents do not leak.

For both of these methods of preparing the aerosol compositions, the active agent generally represents from about 0.1% to about 30% by weight of the total aerosol composition. In one embodiment, the active agent represents about 1 wt % to about 5 wt % of the total composition.

2. Liquid Formulations

The formulations of the present invention may also take the form of a liquid composition for nasal inhalation. Liquid formulations are well known in the art. See, for example, Remington: The Science and Practice of Pharmacy, supra. It is preferred that the liquid is an aqueous suspension, although aqueous solutions may be used as well. The liquid formulations may include one or more carriers in addition to the active agent. Generally, the carrier is a sodium chloride solution having a concentration such that the formulation is isotonic relative to normal body fluid. In addition to the carrier, the liquid formulations may contain water and/or excipients including an antimicrobial preservative (e.g., methylparaben, propylparaben, butylparaben, benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), and/or a surfactant (e.g., Poloxamer, PEG 40 Stearate, polysorbate 80, sodium lauryl sulfate, sorbitan monopalmitate and combinations thereof). Combining the components followed by conventional mixing, results in a liquid formulation suitable for nasal delivery. Typically, the active agent will make up from about 0.1% to 20% by weight of the total liquid composition. In one embodiment, the active agent represents about 0.5 wt % to about 5 wt % of the total composition.

When in an aqueous solution, the present formulations may optionally contain a polymeric carrier and/or therapeutic extender, as described, for example, in U.S. Pat. No. 6,316,483, to Hanswalter et al.

3. Dry Powder Formulations

The dry powder formulations as described herein include, at a minimum, the calcium channel blocker. Such dry powder formulations can be administered by nasal inhalation to a patient without the need of a carrier. Preferably, dry powder formulations that do not include a carrier are administered with the aid of, for example, a dry powder inhaler.

Preferably, however, the dry powder formulations described herein include one or more pharmaceutically acceptable carriers. Although any carrier suitable for nasal drug administration may be used, pharmaceutical sugars are particularly preferred for use as carriers in the present invention. Preferred pharmaceutical sugars include those selected from the group consisting of fructose, galactose, glucose, lactitol, lactose, maltitol, maltose, mannitol, melezitose, myoinositol, palatinite, raffinose, stachyose, sucrose, trehalose, xylitol, as well as hydrates thereof, and combinations of any of the foregoing. In preferred embodiments, lactose, e.g., lactose U.S.P., serves as the carrier when the formulation is a dry powder.

Once selected, the active agent, or a combination of active agents, is blended to form a substantially homogeneous powder mixture. Techniques involved with the preparation of such powders are well known in the art. Briefly stated, however, the preparation generally includes the steps of reducing the particle size of each active agent (again, alone or in combination), and blending. Of course, reducing the particle size of each active agent is not required when a commercially available product having a suitable particle size is used. Techniques for reducing the particle size include, for example, using mills such as an air-jet mill or a ball mill. It is desirable that the active agent has a particle size diameter of between about 0.1 μm to about 65 μm for nasal administration. It is preferred that the active agent particles are about 1 μm to about 10 μm, more preferably about 2 μm to about 5 μm in diameter.

Similarly, the particle size of the remaining components (e.g., carrier, excipient, etc.) must be controlled. The same techniques described above for reducing the particle size of active agents may be used to reduce the particle size of the remaining components. Again, such techniques are not required when the component is available commercially in the desired particle size range. Preferably, the remaining components, particularly the carrier, have a particle size from about 30 μm to about 160 μm in diameter, with sizes from about 30 μm to about 70 μm most preferred.

For any given particle size range, it is preferred that at least about 60%, more preferably at least about 70%, still more preferably at least about 85%, of the stated particles have a size within the stated or given range. It is most preferred, however that at least about 90% of the particles have the size in the stated or given range. For example, when a component is stated to have a particle size less than 10 μm, it is most preferred that at least 90% of the particles of that component have a particle size of less than 10 μm.

As noted above, some components of the formulation may be commercially available in the desired particle size range. For example, a preferred lactose product for use in some embodiments of the present invention is the PHARMATOSE™ 325 brand of lactose monohydrate available from DMV International, Veghel, The Netherlands. According to the manufacturer, 100% of the lactose particles have a particle size of less than 100μm, and only 5 to 10% of the particles have a particle size of less than 32 μm. Furthermore, a minimum of 70% of the lactose particles are stated to have a particle size of less than 63 μm. Advantageously, particle size manipulation steps are avoided when components are commercially available in the desired particle size range.

Preferably, the particle size reduction of the active agent and the particle size reduction of the remaining components are carried out separately. In this way, it is possible to provide a formulation in which the particle size of the active agent is smaller than the particle size of, for example, the carrier. The advantage of such a formulation is that the active agent penetrates deeply into the nasal passages, while the carrier (having a relatively larger particle size) is retained deep within the nasal passages.

Conventional blending techniques known to those skilled in the art may be used for combining more than one active agent or for combining the active agent with the carrier and/or remaining components. Such blending techniques include passing the combined powders through a sifter or blending, for example, the active agents and carrier in a powder blender such as a “double cone” blender or a “V-blender.” No matter which technique is employed, however, it is necessary that the resulting powder is a substantially homogeneous mixture. Typically, the active agent will make up from about 1% to about 50% by weight of the total dry powder composition. In one embodiment, the active agent represents about 2 wt % to about 20 wt % of the total composition.

After blending, the powder formulation may, if desired, be portioned and/or otherwise processed into unit dose quantities, e.g., portioned into unit dose quantities and individually placed within a unit dosage form or drug delivery system. Alternatively, the powder formulation may be loaded into a dosage form or drug delivery device and not “metered out” into unit doses until used. Accordingly, the invention encompasses dosage forms containing a pharmaceutical composition formulated for nasal drug administration comprising therapeutically effective amounts of a calcium channel blocker.

C. Other Ingredients

The pharmaceutical compositions of the present invention may further comprise one or more excipients, such as bioadhesive materials and permeation enhancers. A therapeutic nasal spray extender such as various gums and polymers can be evaluated to determine their suitability as a bioadhesive material to extend the nasal muco-cilia clearance time of nasal spray formulations. Desired properties of a bioadhesive material include solubility, clarity and compatibility in a conventional nasal spray formulation. In addition, the nasal spray formulation containing the bioadhesive material can be evaluated to determine the concentration effect on spray pattern and resultant mist properties. Suitable bioadhesive extenders include, for example, Carbopol, methylcellulose, hydroxypropyl methylcellulose, hydroxyethylcellulose, hydroxypropylcellulose, sodium carboxymethylcellulose, polyvinyl alcohol, polyvinylpyrrolidone, polyacrylates, polyacrylamide, dextran, gellan gum, poloxamer, calcium polycarbophil, cellulose acetate phthalate, sodium hyaluronate, hyaluronic acid, alginate, chitosan, and so forth.

The formulations of the present invention can also contain a permeation enhancer. Since the inherent permeability of the nasal passages to some drugs may be too low to allow therapeutic levels of the drug to pass through the nasal skin, it may be helpful to co-administer a skin permeation enhancer with such drugs. Suitable enhancers are well know in the art and include, for example, bile salt derivatives, dimethylsulfoxide (DMSO), dimethyl formamide (DMF), N,N-dimethylacetamide (DMA), decylmethylsulfoxide (C₁₀MSO), C₂-C₆ alkanediols, and the 1-substituted azacycloheptan-2-ones, alcohols, and the like.

The formulations of the present invention can optionally contain moisturizing agents such as propylene glycol, glycerin, water soluble polyethylene glycol (PEG) polymers and the like. Preferably, polyethylene glycol is used and is commercially available in average molecular weights ranging from about 200 to greater than 20,000. The commercially available grades of polyethylene glycol are marketed based on the average molecular weight, i.e. the grade nomenclature is identified with the molecular weight. For example, PEG 400 represents material with an average molecular weight of 400 and the material with an average molecular weight of 600 is known as PEG 600. PEG 200, 300, 400, and 600 are clear viscous liquids at room temperature, while PEG 900, 1000, 1450, 3350, 4500 and 8000 are white, waxy solids. Preferred polyethylene glycols for the compositions of this invention are the short to medium chain PEG polymers such as PEG 400 to PEG 3350, and the most preferred polyethylene glycol is PEG 1450. The amount of moisturizing agent that can optionally be present in the compositions of this invention is from about 0.00 to 15.0% by weight/volume of the total composition. Ranges of 0.5% to 10% by weight/volume of the total composition are particularly suitable and a range of 2.5 to 5% by weight/volume is most preferable.

The compositions of the present invention can also optionally contain at least one antimicrobial preservative in the range of 0.001% to about 0.3% by weight/volume of the composition. Examples of such antimicrobial preservatives include benzalkonium chloride, cetylpyridinium chloride, cetylpyridinium bromide, chlorobutanol, chlorhexidine acetate, chlorhexidine HCl, chlorhexidine digluconate, chlorocresol, methylparaben, propylparaben, butylparaben, phenoxyethanol, phenylmercury salts, sorbic acid, thiomersal.

The formulations of the present invention can also optionally include pharmaceutically acceptable buffers sufficient to adjust and maintain the pH of the compositions of the present invention in the range of about 3.0 to about 8.0, preferably about 4.0 to about 5.0. Suitable buffers include citrate, phosphate, tromethamine, glycine, borate or acetate salts, which can also be derived from substances of the type such as citric acid, primary or secondary sodium phosphate, glycine, boric acid, sodium tetraborate, acetic acid and sodium acetate. Other excipients such as hydrochloric acid or sodium hydroxide can also be used for pH adjustment.

Other excipients can be used for adjusting the tonicity or osmolality of the formulations, such as sodium chloride, potassium chloride, mannitol, glucose, sorbitol, glycerol or propylene glycol in concentrations of from about 0.1% to about 20% by weight of the total composition.

D. Tailored Release Profiles

The compositions of the present invention can be formulated to provide “controlled release” of the active agent, wherein release of the drug is not immediate. That is, with a “controlled release” formulation, administration does not result in immediate release of the drug into an absorption pool. The term is used interchangeably with “nonimmediate release” as defined in Remington: The Science and Practice of Pharmacy, Twentieth Ed. (Easton, Pa.: Mack Publishing Company, 2000). In general, the term “controlled release” as used herein includes delayed release formulations, which is optimally intended to prolong the time of a therapeutic effect for an active agent.

The compositions of the present invention can also be formulated to provide “sustained release” (synonymous with “extended release”), which is used in its conventional sense to refer to a drug formulation that provides for gradual release of a drug over an extended period of time. Preferably, although not necessarily, “sustained release” results in substantially constant blood levels of a drug over an extended time period.

The compositions of the present invention can further be formulated to provide a “timed release” of the active agent, wherein release of the drug occurs in a burst or bursts. Timed release, therefore, results in providing the patient with a brief, sudden increase in an otherwise constant amount of the active agent. Thus, a “burst” of an active agent results in a sudden release of a desired amount of an active agent from a delivery system such that as a result of this release, there is a rapid increase in the concentration of the active agent at the desired site in the patient. Such increase is above whatever level of the active agent had been previously present, if any, prior to the burst. The increase is not sustained in a prolonged fashion unless repeated bursts are provided. Preferably, the burst is the result of an immediate release or a short sustained release of the drug.

The compositions of the invention can be formulated into the any of the aforementioned controlled release, sustained release or timed release formulations using methodologies that are well known in the art.

### III. UTILITY AND ADMINISTRATION

The present invention provides a method of increasing the amount of a calcium channel blocker delivered to the brain of a patient suffering from hypertension by nasally administering a pharmaceutical composition comprising a therapeutically effective amount of the calcium channel blocker.

The present invention also provides a method of treating a patient suffering from hypertension by targeting delivery of a calcium channel blocker to the brain of the patient by administering the calcium channel blocker intranasally.

Exemplary cardiovascular disorders for which these methods find utility, include hypertension, heart failure, stroke, angina pectoris, arrhythmias, and arteriosclerosis including coronary artery disease. Methods of treating hypertension are particularly preferred. The compositions are effective in the treatment of patients suffering from both acute and chronic episodes of these maladies.

By using compositions of the present invention to treat patient with hypertension, patients are expected to experience more effective treatment of the disease because the amount of calcium channel blocker that reaches the brain is maximized. Further, by using an intranasal formulation, the calcium channel blocker reaches the brain quicker and thus, less drug can be used in the formulation as compared with commercial oral dosage forms. Accordingly, one embodiment of the invention is an improved method of treating a cardiovascular disorder with a calcium channel blocker; the improvement comprising administering the calcium channel blocker nasally to maximize the amount of calcium channel blocker reaching the brain and to minimize peripherally mediated adverse events.

In a preferred embodiment, for use in the methods of the invention, the calcium channel blocker is selected from verapamil, diltiazem, cinnarizine and nifedipine. Typically, the composition will be administered in a unit dosage form containing the calcium channel blocker in an amount of about 0.2 to about 20 mg of the composition, and will be administered at least one, and up to several times, daily. The duration of treatment will be for as long as needed. Since many cardiovascular disorders can be chronic in nature, patients may receive treatment for many years, and even for a lifetime.

The actual amount of the active agents used the present compositions will, of course, depend upon the age, weight, and general condition of the subject, the severity of the condition being treated, and the judgment of the prescribing physician. Therapeutically effective amounts of the active agent are known to those skilled in the art and/or are described in the pertinent reference texts and literature. In a preferred embodiment, the methods and compositions of the invention will achieve the desired therapeutic effect with a lower dosage than is currently being used. An effective amount of the formulation may be administered with a single administration, e.g., serially administration of the contents of a single capsule containing a therapeutically effective amount of the formulation by a dry powder inhaler or a single actuation of an aerosol inhaler designed to deliver a therapeutically effective amount of the formulation. Alternatively, a patient can obtain an effective amount of the formulation by, for example, administering multiple doses, e.g., serially administering the contents of multiple capsules containing the formulation by a dry powder inhaler.

For the active agents that have known pharmacological profiles and dosing regimens (e.g., as referenced in the Physician's Desk Reference), one skilled in the art can readily determine appropriate dosages for use in the methods and compositions of the invention.

The compositions may be administered in a variety of dosing regimens including: as-needed administration; one, two, three or four administrations once daily; one, two, three or four administrations twice daily; one, two, three or four administrations three times daily; and one, two, three or four administrations four times daily.

The compositions of the invention may be administered by nasal inhalation or by use of a propellant system to deliver the composition to the nasal passages and/or nasal sinuses. The inhaled composition progressively comes into contact with the air passages of the nasal passages, cranial sinuses, and throat area, e.g., the upper respiratory tract. During nasal inhalation, the patient inhales the composition through the nares, either one at a time or simultaneously. For example, the composition may be administered by a pump spray in which the patient administers a spray or powder in the left nare followed by administration in the right nare, or by using a spray or powder inhaled in both nares simultaneously. Alternately, a medicine dropper can be used to dispense a liquid formulation.

In a preferred embodiment, the compositions described above have been formulated to obtain the desired daily dosages by administration of the compositions from one to four times daily, as needed. Of course, the individual daily doses necessary will vary depending upon a particular patient and the severity of the patient's condition, but tailoring a daily dosage to a particular patient is well within the ordinary skill of a practicing physician or pharmacist in this field. Particular modes of nasal administration and devices for delivering meter doses by powder, spray, aerosol, or other liquid application methods are well known in this field to the ordinary practitioner.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents and publications mentioned herein are hereby incorporated by reference in their entireties.

The practice of the present invention will employ, unless otherwise indicated, conventional techniques of pharmaceutical formulation, medicinal chemistry and the like, which are within the skill of the art. Such techniques are explained fully in the literature. Preparation of various types of pharmaceutical formulations are described, for example, in Remington: The Science and Practice of Pharmacy, 20^{th} edition (Lippincott Williams & Wilkins, 2000) and Ansel et al., Pharmaceutical Dosage Forms and Drug Delivery Systems, 6^{th} Ed. (Media, Pa.: Williams & Wilkins, 1995).

The following examples are put forth so as to provide those of ordinary skill in the art with a complete disclosure and description of how to make and use the compositions of the invention. Efforts have been made to ensure accuracy with respect to numbers (e.g., amounts, temperature, etc.) but some experimental error and deviations should, of course, be allowed for. Unless indicated otherwise, parts are parts by weight, temperature is degrees centigrade and pressure is at or near atmospheric. All components were obtained commercially unless otherwise indicated.

### EXAMPLES

### Example 1

### Efficacy Testing

In the following tests, anti-hypertensive formulations are delivered via the intranasal delivery route. A series of dosages and active agents including diltiazem, verapamil, cinnarizine, and nifedipine were tested for their effects on the reduction of blood pressure in laboratory animals. Lab animals were subjected to activity tests, both roto-rod and open field locomotion analysis to determine if the active agents and mode of delivery affected physical activity.

### Diltiazem

In a series of experiments, different groups of male Sprague-Dawley rats (Charles River Laboratories, Wilmington, Mass.) were used. Mean arterial blood pressure was measured using a Digi-Med Blood Pressure Analyzer. The rats were anesthetized with urethane (2 g/kg, IP) and the left carotid artery was cannulated. A baseline blood pressure was recorded then the drug (or vehicle) was administered and blood pressure recorded for 45-60 minutes.

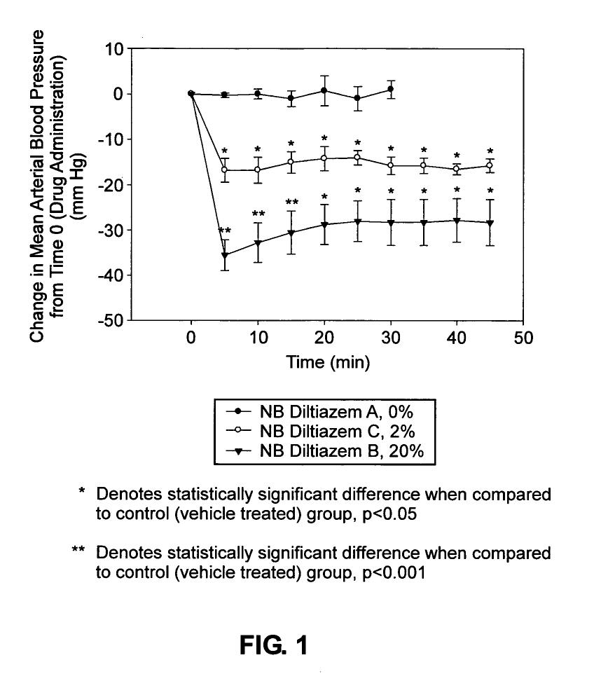

As seen in FIG. 1, three groups of male Sprague-Dawley rats, 4 animals per group, were used in this experiment. A baseline blood pressure was recorded for 20 minutes, then diltiazem and the vehicle or the vehicle alone (control) was administered nasally (20 uL/nare of 0, 2, and 20% solutions). There was a statistically significant difference between the drug treated groups when compared to the vehicle treated group (ANOVA, Student Newman Keuls Test). The vehicle used in this experiment was an isotonic buffer, pH 3.5.

### Diltiazem v. Verapamil

Three groups of male Sprague-Dawley rats, 6 animals per group, were used in this experiment. A baseline blood pressure was recorded for 20 minutes then a vehicle (distilled water), diltiazem 2%, or verapamil 2% were administered nasally (20 uL/nare). There was a statistically significant difference between the drug treated groups when compared to the vehicle treated group.

### Cinnarizine v. Nifedipine

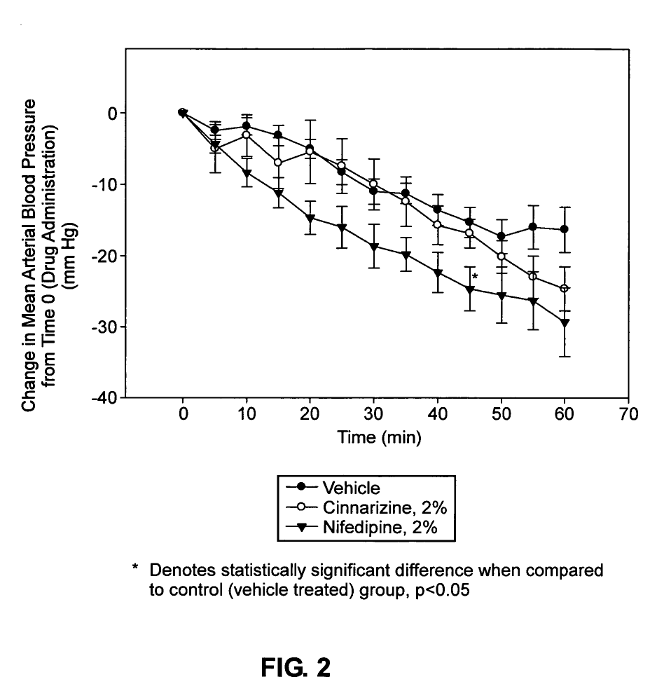

As seen in FIG. 2, three groups of male Sprague-Dawley rats, 7 animals per group, were used in this experiment. A baseline blood pressure was recorded for 20 minutes then a vehicle (cyclodextrin), cinnarizine 2%, or nifedipine 2% were administered nasally (20 uL/nare). Only the nifedipine group at 45 minutes was statistically significantly different from the vehicle treated group.

### Microdialysis

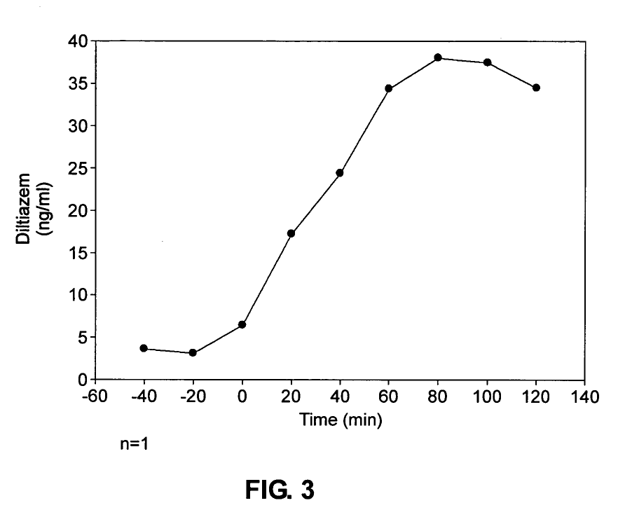

Male Sprague-Dawley rats, 450-500 g, 3 animals per group (Charles River Laboratories, Wilmington, Mass.), were used in this experiment. The rats were anesthetized with urethane (2 g/kg, IP) and microdialysis probes were placed in their left striatum. Baseline microdialysates were collected for 60 minutes, then diltiazem (20% solution) was administered nasally (20 uL/nostril at time 0 minutes, FIG. 3) and microdialysates were collected for 120 minutes (6 samples for 20 minutes each).

As seen in FIG. 3, HPLC analysis of the microdialysates for diltiazem showed a statistically significant difference between the samples collected after drug administration (except the first sample, collected after 20 minutes) and those collected before drug administration (baseline samples).

### Other Testing

Different groups of male Sprague-Dawley rats (Charles River Laboratories, Wilmington, Mass.) were used in the following experiments. Rats were placed individually in a 50″×50″ field (Plexiglas chamber) to measure the distance traveled and velocity. A video tracking system (EthoVision) was used to record the rat movement and to analyze the recorded path. Each rat went through 2 sessions (5 minutes each): a control session 1, before drug administration, and a test session 2, an hour after drug administration. The drug effect on the total distance traveled per session was expressed as the percentage of change between the 2 sessions [(distance traveled in session 2/distance traveled in session 1) %].

### Effect of Nasal Administration of Different Concentrations of Diltiazem (Benzothiazepine Derivative) on the Distance Traveled by Rats in Open Field Locomotion Test

Four groups of male Sprague-Dawley rats, 175-200 g, 6 animals per group, were used in this experiment. Diltiazem was administered nasally (20 uL/nare of 0, 2%, and 20% solutions). The fourth group was a control group where the rats were only handled and nothing was administered nasally in order to exclude the possible effect of handling on locomotion.

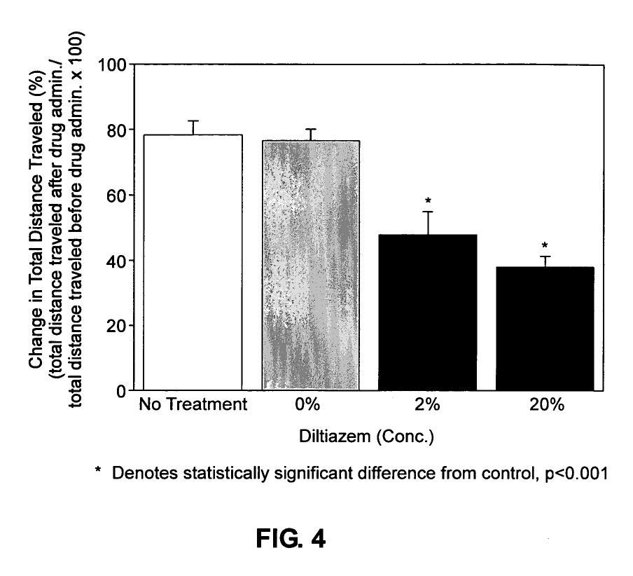

As seen in FIG. 4, there was a statistically significant difference between the drug treated groups when compared to the vehicle treated and no treatment groups. The vehicle used in this experiment was an isotonic buffer, pH 3.5.

### Effect of Nasal Administration of Different Calcium Channel Blockers (Diltiazem; A Benzothiazephine Derivative, and Verapamil; A Phenylalkylamine) on the Distance Traveled by Rats in Open Field Locomotion Test

Four groups of male Sprague-Dawley rats, 175-200 g, 7 animals per group, were used in this experiment. A vehicle (distilled water), diltiazem 2%, or verapamil 2% were administered nasally (20 uL/nare). The fourth group was a control group where the rats where just handled and nothing was administered nasally in order to exclude the possible effect of handling on locomotion.

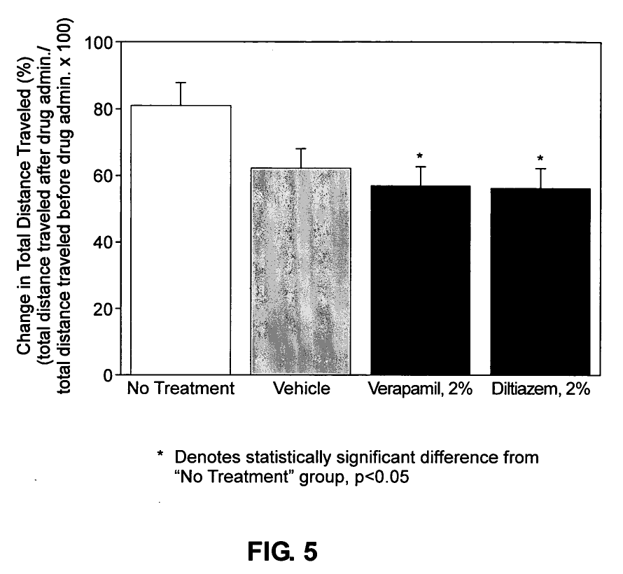

As seen in FIG. 5, there was a statistically significant difference between the drug treated groups when compared to the “no treatment” group; however, there was no significant difference between the drug treated groups and the vehicle treated group.

### Effect of Nasal Administration of Different Calcium Channel Blockers (Cinnarizine; A Piperazine Derivative, and Nifedipine; A Dihydropyridine) on the Distance Traveled by Rats in Open Field Locomotion Test

Four groups of male Sprague-Dawley rats, 175-200 g, 7 animals per group, were used in this experiment. A vehicle (cyclodextrin), cinnarizine 2%, or nifedipine 2% were administered nasally (20 uL/nare)). The fourth group was a control group where the rats where just handled and nothing was administered nasally in order to exclude the possible effect of handling on locomotion.

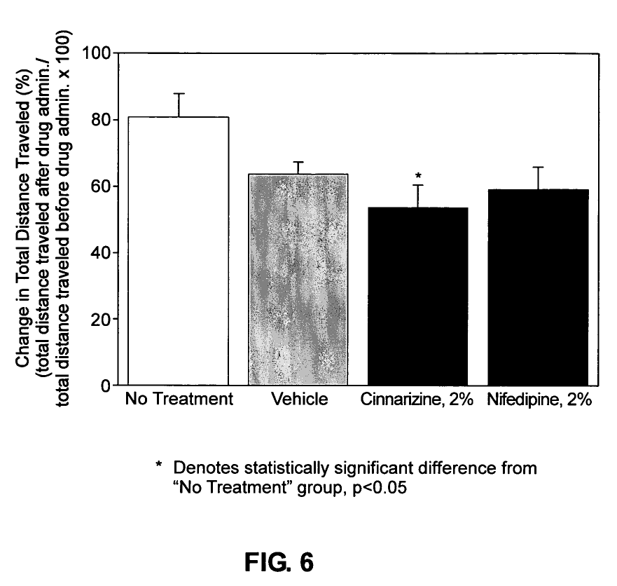

As seen in FIG. 6, there was a statistically significant difference between the cinnarizine treated group when compared to the “no treatment” group, however, there was no significant difference between the drug treated groups and the vehicle treated group.

### Example 2

### Nasal Spray or Drops Containing Diltiazem

Purified water (4.5 Kg) is placed into a suitable stirrer-equipped container. Diltiazem (2.5 g), hydroxymethylcellulose (5 g), sodium edentate (2.5 g), methylparaben (9.0 g), propylparaben (1.0 g), and sorbitol solution (70%, 333.3 g) are added. Enough purified water is added to bring the volume to 4.95 L, and then the solution pH is adjusted to a pH of 5.0 by adding pharmaceutical grade 1 N sodium hydroxide. More purified water is added with stirring, in an amount sufficient to give a final solution volume of 5 L. The solution is filtered through a membrane filter having a pore size of 0.2 μM and dispense into pump spray bottles (such as the spray bottle disclosed in the Schering Corporation Industrial Design Deposit DM/026304, registered Jun. 1, 1993). Such a solution and metering device is expected provide the appropriate dose (one or two sprays into each nare of the nasal passages) for once or twice daily administration.

### Example 3

### Nasal Spray or Drops Containing Nifedipine

Purified water (4.5 Kg) is placed into a suitable stirrer-equipped container. Nifedipine (2.5 g), hydroxymethylcellulose (5 g), sodium edentate (2.5 g), methylparaben (9.0 g), propylparaben (1.0 g), and sorbitol solution (70%, 333.3 g) are added. Enough purified water is added to bring the volume to 4.95 L, and then the solution pH is adjusted to a pH of 5.0 by adding pharmaceutical grade 1 N sodium hydroxide. More purified water is added with stirring, in an amount sufficient to give a final solution volume of 5 L. The solution is filtered through a membrane filter having a pore size of 0.2 μM and dispense into pump spray bottles (such as the spray bottle disclosed in the Schering Corporation Industrial Design Deposit DM/026304, registered Jun. 1, 1993). Such a solution and metering device is expected provide the appropriate dose (one or two sprays into each nare of the nasal passages) for once or twice daily administration.

### Example 4

### Nasal Spray or Drops Containing Verapamil

Purified water (4.5 Kg) is placed into a suitable stirrer-equipped container. Verapamil (2.5 g), hydroxymethylcellulose (5 g), sodium edentate (2.5 g), methylparaben (9.0 g), propylparaben (1.0 g), and sorbitol solution (70%, 333.3 g) are added. Enough purified water is added to bring the volume to 4.95 L, and then the solution pH is adjusted to a pH of 5.0 by adding pharmaceutical grade 1 N sodium hydroxide. More purified water is added with stirring, in an amount sufficient to give a final solution volume of 5 L. The solution is filtered through a membrane filter having a pore size of 0.2 μM and dispense into pump spray bottles (such as the spray bottle disclosed in the Schering Corporation Industrial Design Deposit DM/026304, registered Jun. 1, 1993). Such a solution and metering device is expected provide the appropriate dose (one or two sprays into each nare of the nasal passages) for once or twice daily administration.

### Example 5

### Nasal Spray or Drops Containing Cinnarizine

Purified water (4.5 Kg) is placed into a suitable stirrer-equipped container. Cinnarizine (2.5 g), hydroxymethylcellulose (5 g), sodium edentate (2.5 g), methylparaben (9.0 g), propylparaben (1.0 g), and sorbitol solution (70%, 333.3 g) are added. Enough purified water is added to bring the volume to 4.95 L, and then the solution pH is adjusted to a pH of 5.0 by adding pharmaceutical grade 1 N sodium hydroxide. More purified water is added with stirring, in an amount sufficient to give a final solution volume of 5 L. The solution is filtered through a membrane filter having a pore size of 0.2 μM and dispense into pump spray bottles (such as the spray bottle disclosed in the Schering Corporation Industrial Design Deposit DM/026304, registered Jun. 1, 1993). Such a solution and metering device is expected provide the appropriate dose (one or two sprays into each nare of the nasal passages) for once or twice daily administration.

### Example 6

### Nasal Spray or Drops Containing Diltiazem

Purified water (3.50 Kg) is placed into a suitable stirrer-equipped container and heated to about 50° C. The following ingredients are individually add to the water with stirring, while maintaining the above temperature: sodium edentate (1.0 g), sodium phosphate dibasic (4.98 g), sodium phosphate monobasic (27.6 g), PVP K-90 (12.5 g), PVP K-30 (50 g) and benzyl alcohol (12.5 g). The mixture is stirred for about 5 min after adding each of the individual ingredients. With continued stirring, the following additional water soluble polymers are individually added: PVP K-90 (12.5 g), PVP K-30 (50 g), and PEG 1450 (125 g), and the mixture is stirred for 5 minutes after the addition of each polymer. With stirring, diltiazem (2.5 g) is added, and the mixture stirred for an additional 5 minutes. With stirring, a solution of methylparaben (9.0 g) and propylparaben (1.0 g) is added, and stirred for at least 5 minutes after the addition is complete. The mixture is cooled to about 30° C. with stirring and then more purified water is added with stirring, in an amount sufficient to give a final solution volume of 5 L. The mixture is stirred until uniform. The solution is filtered utilizing conventional filtering equipment and dispense into pump spray bottles (such as the spray bottle disclosed in the Schering Corporation Industrial Design Deposit DM/026304, registered Jun. 1, 1993). Such a solution and metering device is expected to provide the appropriate dose (one or two sprays into each nare of the nasal passages) for once or twice daily administration.

### Example 7

### Sustained Delivery Nasal Spray Containing Diltiazem

The same procedures were followed with the same proportions as in Example 6, above. Such a solution and metering device will provide the appropriate dose (one or two sprays into each nare of the nasal passages) for once or twice daily administration, and will provide a sustained release type composition for administration.

### Example 8

### Dry Pharmaceutical Composition Containing Cinnarizine

Cinnarizine (10.0 mg) and lactose (2000 mg) are blended using conventional blending techniques to form a dry pharmaceutical composition. The dry composition is then placed, in equal portions, into 100 capsules (capsule size 4). Such capsules may be pierced to dispense a unit dose into a nasal inhaler for such dry powder administration. Other dispensing devices may be utilized without forming capsules, for example one may wish to use a dry volume dispensing device that can be rotated to dispense from a larger chamber into a smaller chamber a measured amount of the dry formulation, followed by closing off the larger chamber by rotating or other means before administering a unit dose from the smaller chamber (or from a third chamber connected therewith).

### Example 9

### Dry Pharmaceutical Composition Containing Nifedepine

Using the same procedures and proportions as in Example 9, similar capsules containing nifedipine (or capsules containing only 50% the dosage amount illustrated in Example 9) are placed in the dry powder inhaler as described in U.S. Pat. No. 5,673,686 to Villax et al. and U.S. Pat. No. 5,881,721 to Bunce et al., except that the inhaler design and inhaler tip has been modified for intranasal administration instead of administration by mouth, i.e., the inhaler tip has been adapted to fit inside the nares of the nasal passages. Once the capsule has been properly aligned and pierced in the inhaler, a patient in need of administration of the compositions according to the present invention holds one nostril closed and the nasal tip of the inhaler into the other nostril nare and inhales strongly through the open nostril to inhale the composition. The inhalation is expected to cause the composition to exit the pierced capsule and travel into the patient's nasal and sinus passages of the upper respirator system.

### Example 10

### Metered-Dose Composition Containing Verapamil

A commercially available aqueous solution (0.05% by weight) of verapamil is obtained. The aqueous solution is then placed in a container that is used to administer a metered dose of verapamil as 2 or 3 sprays (such as the spray bottle disclosed in the Schering Corporation Industrial Design Deposit DM/026304, registered Jun. 1, 1993). The solution is administered nasally every twelve hours as 3 to 4 sprays to each nostril with such a metering device to provide a therapeutically effective dose of the composition.

### Example 11

### Aerosol Pharmaceutical Composition

The active component, e.g., diltiazem, is placed into an empty aerosol container. Thereafter, a valve assembly is inserted into the aerosol container and crimped into place so as to provide an airtight seal. The propellant/carrier is then metered through the valve assembly from a tank of bulk propellant/carrier stored under pressure. The aerosol container is then placed in an adaptor suited for actuating aerosol containers and delivering metered amounts of the active agents to a patient.

1. A method of increasing the amount of a calcium channel blocker delivered to the brain of a patient suffering from hypertension, comprising nasally administering a pharmaceutical composition comprising a therapeutically effective amount of the calcium channel blocker.

2. The method of claim 1, wherein the calcium channel blocker is selected from verapamil, diltiazem, cinnarizine, and nifedipine.

3. The method of claim 1, wherein the calcium channel blocker represents about 0.2% to about 30% by weight of the composition.

4. The method of claim 3, wherein the calcium channel blocker represents about 2% to about 20% by weight of the composition.

5. The method of claim 1, wherein the composition further comprises a pharmaceutically acceptable carrier that is suitable for nasal drug administration.

6. The method of claim 5, wherein the composition is administered in the form of an aerosol nasal spray.

7. The method of claim 6, wherein the carrier is a propellant.

8. The method of claim 5, wherein the composition is administered in the form of a liquid.

9. The method of claim 8, wherein the carrier is purified water, saline, buffer, or a combination of any of the foregoing.

10. The method of claim 1, wherein the composition is administered in the form of a dry powder.

11. The method of claim 5, wherein the composition is administered in the form of a dry powder.

12. The method of claim 11, wherein the carrier is selected from fructose, galactose, glucose, lactitol, lactose, maltitol, maltose, mannitol, melezitose, myoinositol, palatinite, raffinose, stachyose, sucrose, trehalose, xylitol, as well as hydrates thereof, and combinations of any of the foregoing.

13. The method of claim 12, wherein the carrier is lactose.

14. The method of claim 1, wherein the composition is administered in a unit dosage form containing the calcium channel blocker in an amount of about 0.2 to about 20 mg of the composition.

15. The method of claim 1, wherein the composition further comprises a therapeutically effective amount of at least one additional pharmaceutically active agent.

16. The method of claim 15, wherein the additional active agents are selected from diuretics, β-blockers, ACE inhibitors, angiotensin II receptor blockers, adrenergic inhibitors, and vasodilators.

17. The method of claim 1, wherein the composition is formulated to provide timed release.

18. The method of claim 1, wherein the composition is formulated to provide sustained release.

19. The method of claim 1, wherein the composition is formulated to provide controlled release.

20. The method of claim 1, wherein the composition further comprises at least one excipient.

21. The method of claim 20, wherein the excipient is a bioadhesive material.

22. The method of claim 21, wherein the bioadhesive material is selected from Carbopol, methylcellulose, hydroxypropyl methylcellulose, hydroxyethylcellulose, hydroxypropylcellulose, sodium carboxymethylcellulose, polyvinyl alcohol, polyvinylpyrrolidone, polyacrylates, polyacrylamide, dextran, gellan gum, poloxamer, calcium polycarbophil, cellulose acetate phthalate, sodium hyaluronate, hyaluronic acid and alginate, chitosan.

23. The method of claim 20, wherein the excipient is a permeation enhancer.

24. A method of treating a patient suffering from hypertension comprising targeting delivery of a calcium channel blocker to the brain of the patient by administering the calcium channel blocker intranasally.

25. An improved method of treating a cardiovascular disorder with a calcium channel blocker; the improvement comprising administering the calcium channel blocker nasally to maximize the amount of calcium channel blocker reaching the brain and to minimize peripherally mediated adverse events.

26. A method of claim 25, wherein the cardiovascular disorder is hypertension.

27. A pharmaceutical composition formulated for nasal drug administration, comprising: a therapeutically effective amount of the calcium channel blocker selected from cinnarizine, and nifedipine.

28. The composition of claim 27, wherein the composition further comprises a pharmaceutically acceptable carrier that is suitable for nasal drug administration.

29. The composition of claim 28, wherein the composition is administered in the form of an aerosol nasal spray.

30. The composition of claim 29, wherein the carrier is a propellant.

31. The composition of claim 28, wherein the composition is administered in the form of a liquid.

32. The composition of claim 31, wherein the carrier is purified water, saline, buffer, or a combination of any of the foregoing.

33. The composition of claim 27, wherein the composition is administered in the form of a dry powder.

34. The composition of claim 28, wherein the composition is administered in the form of a dry powder.

35. The composition of claim 34, wherein the carrier is selected from fructose, galactose, glucose, lactitol, lactose, maltitol, maltose, mannitol, melezitose, myoinositol, palatinite, raffinose, stachyose, sucrose, trehalose, xylitol, as well as hydrates thereof, and combinations of any of the foregoing.

36. The composition of claim 27, wherein the composition further comprises a therapeutically effective amount of at least one additional pharmaceutically active agent.

37. The composition of claim 27, wherein the composition is formulated to provide timed release.

38. The composition of claim 27, wherein the composition is formulated to provide sustained release.

39. The composition of claim 27, wherein the composition is formulated to provide controlled release.

40. The composition of claim 27, wherein the composition further comprises at least one excipient.

41. The composition of claim 40, wherein the excipient is a bioadhesive material.

42. The composition of claim 40, wherein the excipient is a permeation enhancer.

43. A nasal administrable drug delivery device, comprising: the composition of claim 28 and a means for housing and dispensing unit dosages of the composition into a nasal passage of a patient.

44. The device of claim 43, comprising a dry powder inhaler, metered-dose inhaler, medicine dropper, or pump spray bottle.
